(19) United States
(12) Patent Application Publication
(10) Pub. No.: US 2010/0227359 A1
(43) Pub. Date: Sep. 9, 2010

(54) System and method for detection of substances

(76) Inventors: Dale F. Tommer, JR., Berkley, MA (US); Klas D. Romberg, Lowell, MA (US)

Correspondence Address: SEYFARTH SHAW LLP, WORLD TRADE CENTER EAST, TWO SEAPORT LANE, SUITE 300, BOSTON, MA, 02210-2028, US

(21) Appl. No.: 11/635,900

(22) Filed: Dec. 8, 2006

(51) Int. Cl. C12Q 1/04; C12M 1/34

(52) U.S. Cl. 435/34; 435/287.3

(57) ABSTRACT

A system, a method, and apparatus for determining the presence of an agent in a sample. The system includes a sampler for collecting the sample, and an attachment means for attaching the sampler to the sample. In addition, the system includes a carrier, wherein the sampler with the sample is substantially aligned with a portion of the carrier. The system also includes an analyzing device which is substantially aligned with the carrier and the sample. Finally, the system includes a chemistry for identifying an indicator of the agent in the sample as determined by the analyzing device.

### FIELD OF THE INVENTION

The present invention relates to the detection of unknown substances, more specifically, to the detection of substances such as endospores using an indicator molecule.

### BACKGROUND OF THE INVENTION

The detection of unknown substances, such as bacterial endospores, is a significant challenge in bioanalytical chemistry. The endospore is a dormant cellular structure in the life cycle of some members of the genera Bacillus and Clostridium. A number of these species can cause deadly disease, food poisoning, or food spoilage, so their detection is important in both commercial and medical sectors.

In U.S. Pat. No. 5,876,960, the technique used to detect bacterial endospores used a marker chemical, e.g., dipicolinic acid (hereinafter “DPA”) present in endospores and complexed it with a lanthanide (terbium or europium). Upon excitation with a UV light (−280 nm), this compound emits light in bands at 490, 545, 590, and 620 nm for the terbium complexes.

The bacterial endospore is most aptly described as a dormant state in the life cycle of certain bacteria (e.g. Clostridium and Bacillus) as depicted in FIG. 1. The bacterium develops this dormant form (endospore) when there is a lack of nutrients or when it is exposed to some other adverse condition that could threaten the cell. In the process of forming the endospore, the vegetative bacterium segregates all the necessary components to restart its life cycle into a small region inside itself called the forespore.

FIG. 1 graphically depicts the process of sporulation (the transition from active, vegetative bacterium to inactive, spore form of the bacterium). The cell 100 initially has its DNA 101 “floating” in the cytoplasm 102 that is contained by the cytoplasmic membrane 103. The outermost layer of the cell is the cell wall 104. When the cell 100 experiences stress 110, such as the lack of food or extreme environmental conditions, it will begin to form a spore. The first step in this process is to isolate the DNA 101 in a region of the changing cell called the forespore 111. The spore cell wall 120 then forms around the DNA 101. Water is ejected from the region within the spore cell wall 120 and the layers of protective organic material begin to build up around the DNA 101, i.e., spore coat formation 130. Once the spore has completely formed, the mother cell 140 usually ruptures liberating the spore 150 into the environment.

Thus, FIG. 1 gives a pictorial view of the process of sporulation. In other words, the endospore continues to form by expelling most of the water from the interior and by surrounding the forespore with several resistant coatings (cortex, inner coat, and outer coat). In most species, the final step is the lysis or destruction of the mother cell 140 that formed the endospore. The final product of this process, the liberated bacterial endospore 150 is a rugged package that contains all the necessary components to reform the vegetative bacterium.

Techniques are known for the detection of bacterial endospores by terbium-dipicolinate photoluminescence, as described in U.S. Pat. No. 5,876,960. This technique uses a marker chemical, DPA.

DPA comprises about 5-20% by weight of the outer shell of bacterial spores, including Anthrax. DPA is unique to bacterial spores and has not been observed in any other organisms, including vegetative cells of spore-forming microbes. Thus, DPA is an indicator molecule for the presence of bacterial spores. When a spore germinates, i.e., converts to a vegetative cell, the spore releases DPA. It is possible to make the spore rapidly shed its case and release the DPA by triggering germination with L-alanine. It has been shown that Terbium (Tb) is able to bond with DPA, forming a photoluminescent complex. When this complex is illuminated with UV light at 250-280 nm, it fluoresces and emits bright green light. This emitted light has a characteristic wavelength signature unique to the Tb(DPA) complex. This light is captured and measured and indicates the presence of DPA and, thus bacterial spores, in a sample.

DPA is not found in coffee creamer, sugar, flour, foot powder, talc powder, sugar and sugar substitutes or other common white powdery substances. Terbium does not react with very many substances to form fluorescent complexes. The combination of the rarity of DPA in the biological world and the unique characteristics of the Tb(DPA) complex makes it extremely selective for bacterial spores and immune to false positive responses. However, highly reliable and/or portable tools having the ability to detect the presence of live bacterial spores on a sample are not readily available. While bench-scale analytical chemistry protocols can be implemented for spore detection, portability and ease of use are issues. Also, such analytical chemistry techniques require a relatively high skill level to practice.

### SUMMARY OF THE INVENTION

In accordance with certain embodiments of the present invention, an apparatus, method, and system for detecting an endospore using an indicator molecule is presented, which is highly portable and reliable. In accordance with the invention, the system and method are implemented in a manner that requires virtually no chemistry skill to practice.

In one embodiment, a sample collecting device or test strip includes four basic components. A first component or probe is used to contain a sample and includes two areas, one of which has an attaching mechanism for attaching the sample. A second component is configured to mate with the first component and includes an opening for accessing the contained sample. A third component supports the first and second components and also has an opening which is substantially aligned with the opening of the second component. In addition, this embodiment includes a fourth component in the form of a pod storing testing chemistry used to identify an indicator.

The sample collecting device or test strip is implemented in a system or method, in an illustrative embodiment wherein a user determines the presence of endospores by providing a testing device including the (sampling) probe which is assembled into the testing strip, and includes an analyzing device that identifies the sample on the probe. The user collects the sample with the probe, substantially aligns the probe and the sample with the testing strip, and interfaces the testing strip and the sample with the analyzing device. Chemistry in the pod on the testing strip is used to identify an indicator molecule, which is tested by the analyzing device.

### BRIEF DESCRIPTION OF THE DRAWINGS

The invention is illustrated in the figures of the accompanying drawings which are meant to be exemplary and not limiting, in which like references are intended to refer to like or corresponding parts, and in which:

FIG. 1 illustrates the process of sporulation in bacterial endospores as known in the art;

FIG. 2 is a block diagram of a detection apparatus in accordance with an embodiment of the present invention;

FIG. 3 is a flow chart depicting a determination of an endospore in accordance with an embodiment of the present invention;

FIG. 4 is a diagram of a detection system including a probe, a test strip and a chemistry pod in accordance with an embodiment of the present invention.

FIG. 5A is a diagram of a front view of the analyzing device for processing a test strip according to the invention; and

FIG. 5B is an illustrative diagram of the inside workings of the analyzing device of FIG. 5A.

FIG. 6 is another embodiment of a detection system including a probe, a test strip and a chemistry pod in accordance with an embodiment of the present invention.

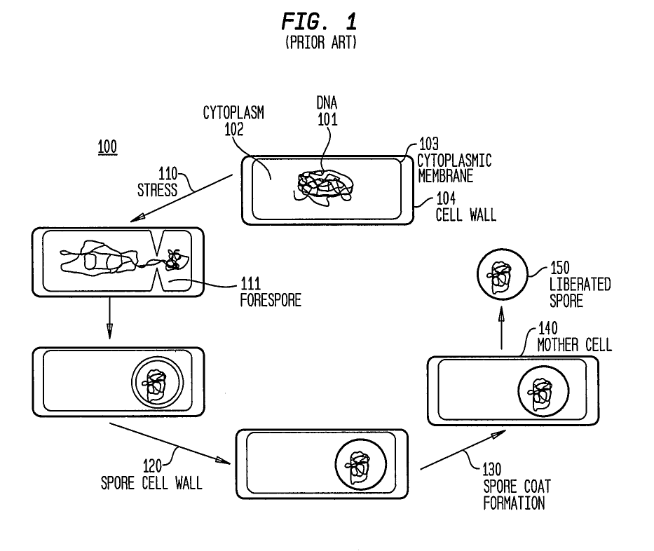

### DETAILED DESCRIPTION

Detailed embodiments of the present invention are disclosed herein, however, it is to be understood that the disclosed embodiments are merely exemplary of the invention, which may be embodied in various forms. Therefore, specific functional details disclosed herein are not to be interpreted as limiting, but merely as a basis for the claims and as a representative basis for teaching one skilled in the art to variously employ the present invention.

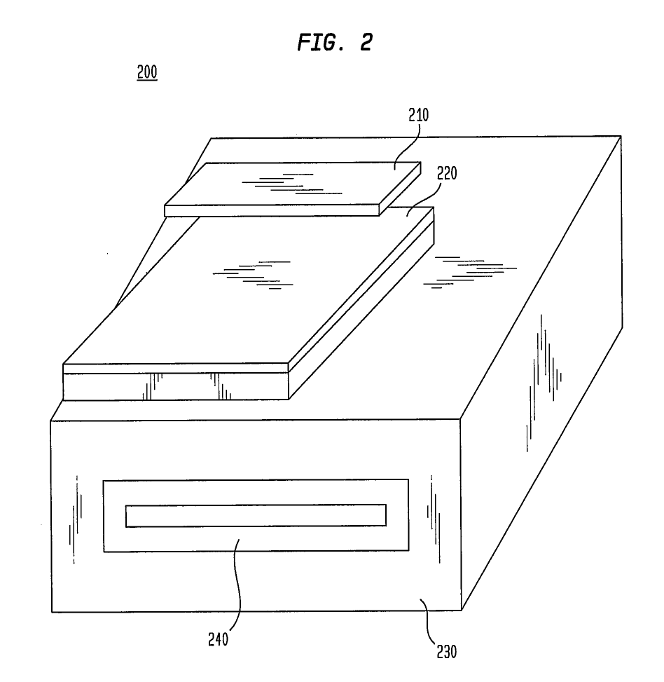

Turning now to FIG. 2, a block diagram of a detection system according to the present invention is depicted. In this illustrative embodiment, an endospore detection system 200 includes three components. The first component, a sampler 210, acts as a probe. The sampler or probe 210 is used to attach to a sample by way of an attaching means, where the sample is to be tested for an agent. The sampler includes an adhesive portion on a surface thereof that is used to adhere and contain the sample. The sampler may be made from any suitable material, such as synthetic polymers and composite materials or any other material that could be permeated by ultraviolet light without loss of signal. The thickness can be relatively thin in the 4-5 mils (0.1-0.125 mm) range, but not limited to that range. Although the sample collection area is described as being circular, it may be made in any shape that is conducive to sample collection.

The length and width of the sampler depends on the dimensions of the analytical apparatus and may be of any suitable size. A suitable adhesive, such as a 3M product should have the following parameters: (1) that it does not cause any distracting fluorescence in ultraviolet light conditions and (2) it also should not migrate to the areas under test. The adhesive will bond to the surface of the probe on the perimeter of the sampler 210. The native adhesive surface of the polymer easily captures dry powdery materials as well as microscopic particles and organisms. The analytical method of the invention is carried out directly on the surface of the probe without transfer procedures needed with consequent loss of sample.

In use in this illustrative embodiment, the sampler 210 is substantially aligned with a carrier 220 on which it is disposed for further processing and chemical analysis. The carrier 220 may be made of plastic or any other durable, rigid material that can be easily handled. The carrier 220, as discussed in greater detail hereinafter, includes a pod (not shown in FIG. 2) that contains the chemistry necessary to yield an indicator molecule of the agent that is being looked for in the sample.

In use, the carrier 220 is also substantially aligned with an analyzing device 230. The analyzing device 230 is a device that facilitates a chemical interaction, such as by causing the chemistry in the pod on the test strip to interact with the sample on the test strip. The analyzing device 230 analyzes, such as by fluorescence detection, whether or not the sample collected by the sampler contains any agents. On an outside wall of the analyzing device 230 is an opening 240, where the sampler 210 disposed on the carrier 220, as appropriately aligned with one another, are inserted. After insertion into the analyzing device 230, a result will be displayed, such as on an LED display or other electronic read-out, for the user to determine whether or not an agent is present within the sample.

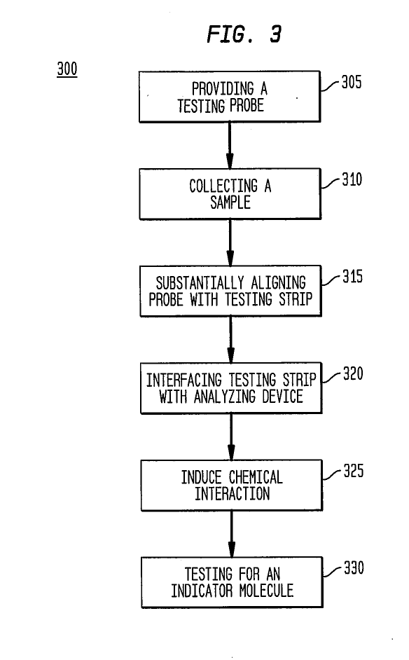

FIG. 3 depicts a flow chart 300 illustrating steps of a method in accordance with an embodiment of the present invention. A sample collector or probe is provided 305 and used to test for the presence of bacterial endospores in a suspicious sample. A user collects the sample 310 using the probe. The method of sample collection using the probe has a number of advantages. The adhesive mechanism of the probe easily captures dry powdery materials as well as microscopic particles and organisms. Once the sample is attached to the probe, it remains on the probe, so no portion of the sample can be lost in the process of transfer between the probe and any other device. In some instances, the sample can be collected from multiple locations on a surface with only one sample probe which thereby serves to concentrate the material to be analyzed.

The probe is substantially aligned with the testing strip 315 to make certain that the sample collected on the probe is properly exposed for the ease and accuracy of testing. The testing strip in this illustrative embodiment contains the pod of chemistry that will be used to interact with the sample to determine the presence of an agent.

The testing strip and pod with the sample aligned thereon is then placed in or otherwise interfaced with the analyzing device 320. The analyzing device generally contains a mechanism, such as rollers to spread the chemistry from the pod, to induce a contained chemical interaction 325 between the sample and the chemistry in the pod. The chemical interaction induced is used to identify an indicator molecule. The indicator molecule, where the substance being tested for is a bacterial endospore, is generally DPA which is used to identify the presence of the bacterial endospore in the sample. The analyzing device tests for the indicator molecule 330 and outputs a result to a user. The chemical interaction and detection for bacterial endospores may be performed in accordance with the techniques described in U.S. Pat. No. 5,867,960 and U.S. patent Application Publication Nos US 2004/0014154 A1; US 2005/0136508 A1 and US 2005/0221418, which are all hereby incorporate by reference herein in their entirety.

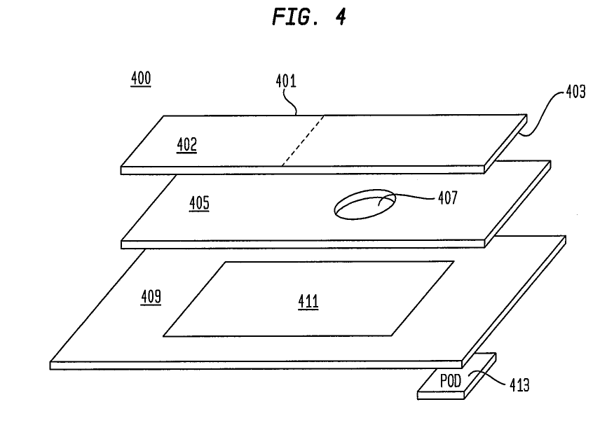

FIG. 4 is a diagram of an embodiment of a testing strip used in an embodiment of the present invention. The testing strip illustrated is for collecting a sample so that the sample can be tested for any of various types of agents. Examples of agents include anthrax and other spore-forming pathogens of the bacillus and clostridium families. For other pathogens, suitable reagents are under development. It should be noted here that the test strip and the apparatus can be used for the detection of any luminescent chemical compound. According to the implementations of the invention, the agent is generally detected by testing for an indicator molecule within the collected sample.

A first member 401 of the testing strip is a probe. The probe is used to collect a sample that is to be tested. The first member, or probe, 401 has a first surface area 402 and a second surface area 403. Either first surface area 402 or second surface area 403, includes a type of attaching mechanism so that the attaching mechanism portion of the probe facilitates attachment of the sample to the probe. The attaching mechanism can be a sticky substance on the surface or a portion of the surface, such as inherent native adhesiveness, or it can be any of various other adhesives. In one embodiment of the present invention, the probe surface is entirely covered with the native characteristic tacky polymeric adhesive. With this adhesive, the suspected sample is attached to the probe without the user having to make contact with the suspected sample. If less than the entire surface has a tacky adhesive, the area that does not have an attaching mechanism is left as such for convenient handling by the user.

The probe 401 can be made from a thin plastic/polymeric material. The sampler probe may also be made from any suitable material such as synthetic polymers and composite materials or any other material that could be permeated by ultraviolet light without loss of signal. The thickness should be relatively thin in the 4-5 mils (0.1-0.125 mm) range, but not limited to such range. Although the sample collection area is described as being circular, it may be made in any shape that is conducive to sample collection. Essentially, the sample collection area can be any size or shape.

In addition, the probe should have no significant characteristic luminescence of its own and should be biologically inactive, i.e. it should neither promote nor inhibit germination of the sample.

The probe should also be environmentally stable and usable in extreme temperature conditions from −40 to +40 degrees Celsius.

The materials used for the probe of the present invention should have appropriate properties such as thickness, stiffness, tear strength, stability, etc. sufficient to perform its function as described herein.

A user can collect a sample of the unknown substance by applying the first member to the sample so that the adhesive portion of the first member comes into contact with the unknown sample, thereby causing at least some of the unknown sample to adhere to the adhesive carried by the first member.

A second member 405 of FIG. 4 has an opening 407. The opening 407 is shown as generally round, but this is only an example and it could be any of various geometric shapes. That is, the opening 407 can be one of several varieties of shapes, for example, a square, a rectangle, or a triangle. The opening can range in size as a function of the form factor of the device. The opening 407 is considered to be a sample collection area, because once the sample has attached itself to the adhesive mechanism of the probe 401, the sample is to be aligned so that the sample is exposed through the opening 407.

A third member 409 of FIG. 4 also has an opening 411. The second opening 411 is shown as generally rectangular, but this too is only an example. The opening 411 can be one of several varieties of shapes, for example, a square, a circle, or a triangle, but it generally should correspond in shape to the opening 407 of the second member 405. The opening 411 is to be aligned with the first opening 407. This way, the sample that is attached to the probe 401 is exposed through both the openings, 407 and 411. In addition, the opening 411 provides a viewing area for exact placement of the probe 401 bearing the sample.

In addition, a pod 413 is included with the testing strip 400. In some embodiments, the pod 413 is a rupturable container filled with a reactive chemical fluid. The pod can also be filled with a liquid chemical, gas or solid. The reactants that participate in the reaction may be included in the fluid in the pod or may be coated on the surface of one or more of the members of the device.

In one embodiment, the pod 413 is a reagent packet containing terbium and L-alanine, such as described in the referenced U.S. Pat. No. 5,876,960. This chemistry mixes with the sample collected on the probe and causes a reaction with the sample. If there is a presence of an agent, there will be an indication of whether there is a presence of DPA.

Any chemistry suited to generate a signal indicating a specific agent being present may be used according to the present invention. Preferred chemistries are those which involve a fluorescent species or that influence a change in the fluorescent properties of one or more chemical species. These include antigen—antibody interactions, fluorescent reactant molecules, derivitization to attach a fluorescent species to another chemical species, quenching of luminescence by the presence of, or reaction with, a chemical species, quantum dot fluorophores and the like.

In other words, the collected sample is mixed with L-alanine and terbium, causing the release of DPA (in a watery environment), which then reacts to form the fluorescent Tb(DPA) complex. The pod containing the L-alanine and terbium is then excited by a pulsed UV source, which is within the analyzing device 230. The resulting luminescence is filtered and focused into the detector. These signals are sampled after a short delay, which permits rejection of rapidly decaying impurity fluorescence and residual light from the pulsed excitation source, resulting in improved detection. When the intense luminescence created by the presence of live spores exceeds a predetermined level, an electronic signal which could be any kind of visual or audible display indicates a positive test result. If no such signal is detected, a negative result is indicated.

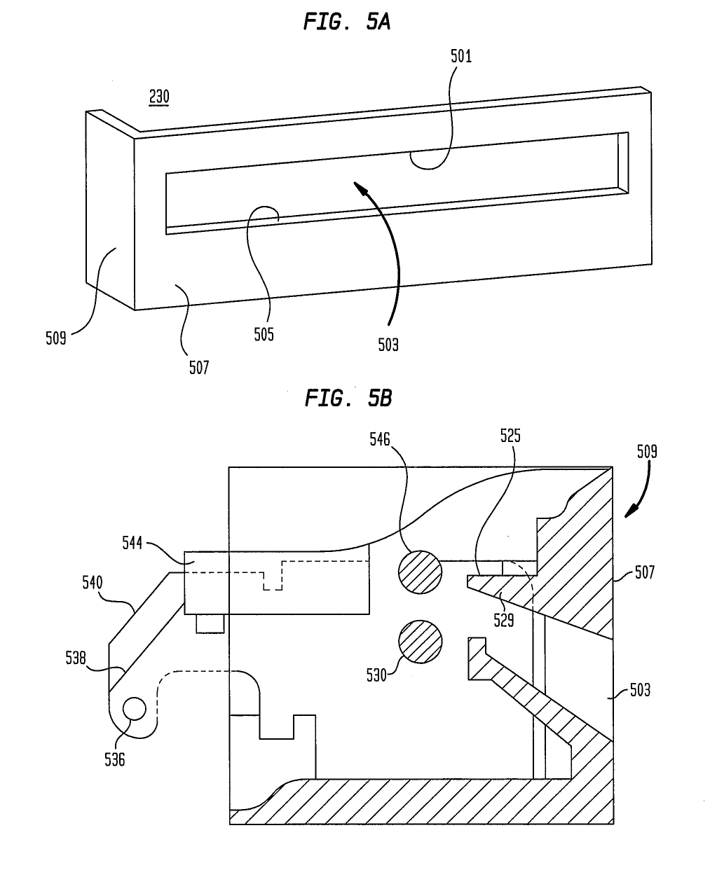

FIG. 5A shows a receiver of an analyzing device for receiving a testing strip. FIG. 5B shows the inside mechanics of FIG. 5A. The receiver 500 includes a housing section 509 having a slot, a door, or any kind of port 503 disposed transversely within a forward wall portion 507 thereof through which a testing strip, with attached probe is introduced into the device 230 to its passage between a pair of juxtaposed pressure-applying members or rollers 546 and 530. The pressure-applying members 546 and 530 apply compressive pressure to the testing strip during its passage between them for distributing a fluid processing composition within the pod to initiate a chemical process within the testing strip. The detection of indicator molecules, as described herein (before), occurs after the compression by the rollers inside of the analyzer. The detection results in a display (not show and as known in the art) indicating the nature of the sample.

The types of displays may be as simple as a red warning light, computerized displays and networked record keeping. The signal may also be processed via wireless communication including live radio and cellular telephone hookups.

As best shown in FIG. 5B, housing section 509 further includes a camming member 525 integrally molded with the forward wall portion 507 and extending rearwardly therefrom for defining a generally planar transverse inclined camming surface 529 which is coextensive with the width of the exit slot 503 and extends rearwardly from the upper edge 501 of exit slot 503 and upwardly therefrom towards an exit side of the pressure-applying members 546 and 530.

The pressure-applying members 546 and 530 should be appropriately located and retained in location within the housing section 509 in order to provide the proper bite between the pressure-applying members 546 and 530 on each side thereof, i.e., the entrance side and the exit side of the compression device. The pressure-applying members apply a compressive pressure along the length and width of the testing strip while advancing it therebetween towards the exit side thereof.

In one embodiment of the present invention, the pressure applying members 546 and 530 apply pressure to the testing strip device along with the pod. The pod, which is filled with a chemical liquid, is broken open because of the pressure applied to it when the pressure-applying members roll over the testing strip and pod. At this point, the sample collected by the probe is exposed to the chemical fluid that was contained in the pod. A chemical interaction or reaction takes place which will indicate whether or not there is a presence of an indicator.

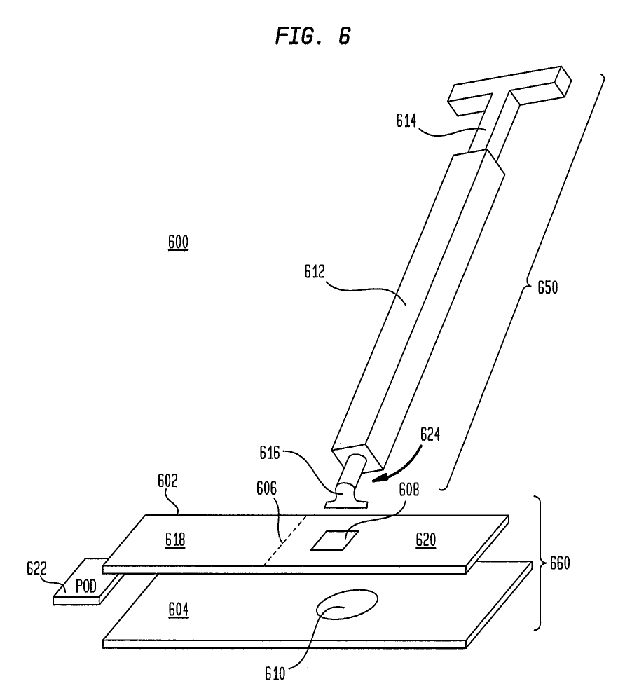

FIG. 6 is a diagram of an alternative embodiment of a testing strip and a handling device used in an alternative embodiment of the present invention. The shown testing strip is for collecting a sample so that the sample can be tested for any type of agent. The agent is generally detected by testing for an indicator molecule within the collected sample.

A pickup strip 602, also called a probe, can be made from a thin plastic material, synthetic polymers and composite materials, or any other material that could be permeated by ultraviolet light without loss of signal. The pickup strip 602 has no significant characteristic luminescence of its own and is biologically inactive. Additionally, the pickup strip 602 is environmentally stable and can be used in extreme temperature conditions ranging from −40 degrees Celsius to 40 degrees Celsius.

This pickup strip 602, used to collect the sample that is to be tested, has at least a first area 618 and a second area 620. Either the first area 618 or the second area 620 includes a type of attaching mechanism so that the attaching mechanism portion of the pickup strip 602 can attach itself to the sample. The attaching mechanism, which can be a sticky substance on the surface or a portion of the surface, attaches itself to the sample to be tested. The attaching mechanism can be an inherent native adhesiveness or any of various other adhesives. In one embodiment of the present invention, the pickup strip 602 is covered with a tacky polymeric adhesive. With this adhesive, the suspected sample is attached to the pickup strip 602 without the user having to make contact with the suspected sample.

In addition, a pod 622 is included with the pickup strip 602. In some embodiments, the pod 622 is a rupturable container filled with a reactive chemical fluid. The pod 622 can also be filled with a chemical gas or solid. The reactants that participate in the reaction may be included in the fluid in the pod 622 or may be coated on the surface of one or more members of the device.

In one embodiment, the pod 622 is a reagent packet containing terbium and L-alanine, such as described in the Referenced U.S. Pat. No. 5,876,960. This chemistry mixes with the sample collected on the probe and causes a chemical reaction with the sample. If there is a presence of an agent, there will be an indication of whether there is a presence of DPA.

The pickup strip 602 and a shield substrate 604 are temporarily attached to one another and form subassembly 660. The shield substrate 604 of FIG. 6 has an opening 610. The opening 610 is shown as generally round, but this is only an example. The opening 610 can be one of several varieties of shapes, for example, a square, a rectangle, or a triangle. The opening 610 can range in size as a function of the form factor of the device.

A user can collect a sample of the unknown by applying the pickup strip 602 and the shield substrate 604, attached to one another 660, to the sample so that the adhesive portion of the pickup strip 602 comes into contact with the unknown sample through the opening 610 in the shield substrate 604, thereby causing at least some of the unknown sample to adhere to the adhesive carried by the pickup strip 602.

The handling device 650 of FIG. 6 is utilized by a user to transport the sample from the sample collecting area to a testing device. The handling device 650 includes a user handle 614, a tube 612, a shaft 624, and a catcher 616.

The handling device 650 attaches to the probe 660 including pickup strip 602 and shield substrate 604 by an attacher 608. The attacher 608, included on the pickup strip 602, attaches to the catcher 616, thereby connecting the probe assembly 660 and the handling device 650.

When a user holds the handle 614 of the handling device 650, which is joined with the tube 612, to lift the probe assembly 660, the attacher 608 and catcher 616 affix to one another. The attacher 608 and the catcher 616 fit together and conform to a locked position.

Once the attacher 608 is affixed to the catcher 616, the probe assembly 660 attaches itself to the handling device 650. After the sample is collected, the shield substrate 604 detaches itself from the pickup strip 602. The user lifts the entire assembly including the pickup strip 602 with the sample attached thereto and draws the pickup strip 602 into the tube 612. The pickup strip 602 folds in half along the perforation 606 and is drawn into the tube 612 so that no part of the sample is exposed. At this point, the pickup strip 602 is ready to be inserted into the analyzer by being released from the handling device 650 and by separating 608 from 602.

While the invention has been described and illustrated in connection with preferred embodiments, many variations and modifications will be evident to those skilled in the art and may be made without departing from the spirit and scope of the invention. The invention is thus not to be limited to the precise details of methodology or construction set forth above as such variations and modification are intended to be included within the scope of the invention.

1. A sample collecting device for testing for an agent comprising:
a first member, including a first area and a second area, at least one of said first area and said second area having an attaching mechanism;
a second member, including a first opening, said first opening being a sample collection area;
a third member, including a second opening, said second opening being substantially in alignment with said sample collection area; and
a pod comprising a testing chemistry to identify an indicator.

2. The sample collecting device of claim 1, further comprising a fourth member interfacing with said pod on a first side with said third member interfacing with said pod.

3. The sample collecting device of claim 1, further comprising a chemical fluid enclosed in said pod.

4. The sample collective device of claim 1, wherein said testing chemistry is a chemical fluid.

5. The sample collecting device of claim 1, wherein said attaching mechanism is an adhesive.

6. The sample collecting device of claim 1, wherein said agent is a bacterial endospore.

7. The sample collecting device of claim 1, wherein said indicator is an indicator molecule for presence of bacterial spores.

8. A method for determining the presence of bacterial endospores in a sample comprising:
providing a testing device including a probe, a testing strip, and an analyzing device;
collecting said sample with said probe;
substantially aligning said probe and said sample with said testing strip;
interfacing said testing strip and said sample with said analyzing device;
providing chemistry for identifying an indicator molecule of said bacterial endospores as determined by said analyzing device; and
testing for presence of said indicator molecule.

9. The method for determining the presence of bacterial endospores of claim 8, further comprising providing a pod with said chemistry and interfacing said pod with said testing strip.

10. The method for determining the presence of bacterial endospores of claim 9, wherein said pod includes a chemical fluid.

11. The method for determining the presence of bacterial endospores of claim 8, further comprising applying pressure to said probe, said testing strip, and said chemistry.

12. A system for determining the presence of an agent in a sample comprising:
a sampler for collecting said sample;
attachment means for attaching said sampler to said sample;
a carrier;
a first interfacing means for substantially aligning said sampler and said sample with a portion of said carrier;
an analyzing device;
a second interfacing means for substantially aligning said carrier and said sample with said analyzing device; and
a chemistry for identifying an indicator of said agent in said sample as determined by said analyzing device.

13. The system for determining the presence of an agent of claim 12, wherein said sampler includes at least a first area and a second area.

14. The system for determining the presence of an agent of claim 13, further comprising at least one of said first area and said second area has an adhesive for attaching said sample.

15. The system for determining the presence of an agent of claim 12, wherein said carrier comprises at least a first opening and a second opening.

16. The system for determining the presence of an agent of claim 15, wherein said first opening and said second opening are substantially aligned.

17. The system for determining the presence of an agent of claim 12, further comprising a pod including said chemistry.

18. The system for determining the presence of an agent of claim 12, wherein said chemistry is a chemical fluid.
